(19) United States
(12) Patent Application Publication
(10) Pub. No.: US 2005/0113566 A1
(43) Pub. Date: May 26, 2005

(54) Inhibitors of C-FMS kinase

(76) Inventors: Mark R. Player, Phoenixville, PA (US); Nand Baindur, Kendall Park, NJ (US); Benjamin Brandt, Philadelphia, PA (US); Naresh Chadha, Montville, NJ (US); Raymond J. Patch, Yardley, PA (US); Davoud Asgari, Plainsboro, NJ (US); Taxiarchis Georgiadis, Carmel, IN (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/970,903

(22) Filed: Oct. 22, 2004

Related U.S. Application Data
(63) Continuation-in-part of application No. 10/831,216, filed on Apr. 26, 2004.
(60) Provisional application No. 60/465,204, filed on Apr. 25, 2003.

(51) Int. Cl. C07D 279/12; C07D 265/30; C07D 223/12

(52) U.S. Cl. 534/551; 540/467; 540/605; 544/59; 544/158; 544/162; 544/382; 546/223; 558/410; 562/450; 562/43

(57) ABSTRACT

The invention relates to compounds of Formula I: 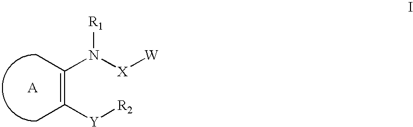 wherein A is
phenyl, naphthyl or biphenyl, each of which may be optionally substituted with one or more of -C₁₋₆ alkyl, amino, aminoalkyl, hydroxyalkyl, alkoxyalkyl, sulfonamidoalkyl, guanidinoalkyl, heteroaryl, halogen, hydroxy, —CF₃, alkoxy, aryl, aralkyl, heteroaralkyl, aryloxy, arylalkoxy, —OCF₃, —OCO-alkyl, —CORA, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b}; or
a 5- to 7-membered mono- or a 8- to 10-membered bicyclic heteroaromatic ring having from one to four heteroatoms selected from N, O or S, and may be optionally substituted with one or more of -C₁₋₆ alkyl, amino, aminoalkyl, hydroxyalkyl, alkoxyalkyl, sulfonamidoalkyl, guanidinoalkyl, heteroaryl, halogen, hydroxy, —CF₃, alkoxy, aryl, aralkyl, heteroaralkyl, aryloxy, arylalkoxy, —OCF₃, —OCO-alkyl, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b};

R₁ is
—H, aryl, —CORₐ, —CORₐ, —COORₐ, —CONRₐR_{b}, —SO₂Rₐ or —SO₂NRₐR_{b}; X is
—CO—, —C(═NH)—, —CS—, —CON(Rₐ)—, —CS(NRₐ)—, —SO₂— or —CRₐR_{b}—; Y is
—S—, —SO—, —SO₂—, —O— or direct link;

R₂ is alkyl, cycloalkyl, heterocyclyl, aryl or heteroaryl, each of which may be optionally substituted with one or more halogens; and W is
phenyl, naphthyl or biphenyl, each of which may be optionally substituted with one or more of C₁₋₄ alkyl, amino, aminoalkyl, hydroxyalkyl, alkoxyalkyl, halogen, hydroxy, —CF₃, alkoxy, aryloxy, arylalkoxy, —OCF₃, —CORₐ, —CN, —C(NH)NH₂, —COORₐ, —CONRₐR_{b}, —NHCORₐR_{b}, —NHSO₂Rₐ, —NO₂, —SORₐ, —SO₃Rₐ or —SO₂NRₐR_{b}; or
a 5- to 6-membered mono- or a 8- to 10-membered bicyclic heterocyclic or heteroaromatic ring having from one to four heteroatoms selected from N, O or S, and may be optionally substituted with -C₁₋₆ alkyl, amino, aminoalkyl, hydroxyalkyl, alkoxyalkyl, heteroaryl, halogen, hydroxy, —CF₃, alkoxy, aryl, aralkyl, heteroaralkyl, aryloxy, arylalkoxy, —OCF₃, —OCO-alkyl, —OCO-alkylamino, —OCO-alkylamido, CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b}; as well as solvates, hydrates, tautomers or pharmaceutically acceptable salts thereof, that inhibit protein tyrosine kinases, especially c-fms kinase.

### RELATIONSHIP TO OTHER APPLICATIONS

This application is a continuation-in-part of U.S. Ser. No. 10/831,216, filed Apr. 26, 2004, now ______; which in turn claimed benefit under 35 U.S.C. § 119(e) to Provisional Application No. 60/465,204, filed Apr. 25, 2003.

### BACKGROUND OF THE INVENTION

The invention relates to novel compounds that function as protein tyrosine kinase inhibitors. More particularly, the invention relates to novel compounds that function as inhibitors of c-fms kinase.

Protein kinases are enzymes that serve as key components of signal transduction pathways by catalyzing the transfer of the terminal phosphate from adenosine 5′-triphosphate (ATP) to the hydroxy group of tyrosine, serine and threonine residues of proteins. As a consequence, protein kinase inhibitors and substrates are valuable tools for assessing the physiological consequences of protein kinase activation. The overexpression or inappropriate expression of normal or mutant protein kinases in mammals has been demonstrated to play significant roles in the development of many diseases, including cancer and diabetes.

Protein kinases can be divided into two classes: those which preferentially phosphorylate tyrosine residues (protein tyrosine kinases) and those which preferentially phosphorylate serine and/or threonine residues (protein serine/threonine kinases). Protein tyrosine kinases perform diverse functions ranging from stimulation of cell growth and differentiation to arrest of cell proliferation. They can be classified as either receptor protein tyrosine kinases or intracellular protein tyrosine kinases. The receptor protein tyrosine kinases, which possess an extracellular ligand binding domain and an intracellular catalytic domain with intrinsic tyrosine kinase activity, are distributed among 20 subfamilies.

Receptor tyrosine kinases of the epidermal growth factor (“EGF”) family, which includes HER-1, HER-2/neu and HER-3 receptors, contain an extracellular binding domain, a transmembrane domain and an intracellular cytoplasmic catalytic domain. Receptor binding leads to the initiation of multiple intracellular tyrosine kinase dependent phosphorylation processes, which ultimately results in oncogene transcription. Breast, colorectal and prostate cancers have been linked to this family of receptors.

Insulin receptor (“IR”) and insulin-like growth factor I receptor (“IGF-1R”) are structurally and functionally related but exert distinct biological effects. IGF-1R over-expression has been associated with breast cancer.

Platelet derived growth factor (“PDGF”) receptors mediate cellular responses that include proliferation, migration and survival and include PDGFR, the stem cell factor receptor (c-kit) and c-fms. These receptors have been linked to diseases such as atherosclerosis, fibrosis and proliferative vitreoretinopathy. These are type III Receptor tyrosine kinase family may or may not be PDGF.

Fibroblast growth factor (“FGR”) receptors consist of four receptors which are responsible for the production of blood vessels, for limb outgrowth, and for the growth and differentiation of numerous cell types.

Vascular endothelial growth factor (“VEGF”), a potent mitogen of endothelial cells, is produced in elevated amounts by many tumors, including ovarian carcinomas. The known receptors for VEGF are designated as VEGFR-1 (Flt-1), VEGFR-2 (KDR), VEGFR-3 (Flt-4). A related group of receptors, tie-1 and tie-2 kinases, have been identified in vascular endothelium and hematopoietic cells. VEGF receptors have been linked to vasculogenesis and angiogenesis.

Intracellular protein tyrosine kinases are also known as non-receptor protein tyrosine kinases. Over 24 such kinases have been identified and have been classified into 11 subfamilies. The serine/threonine protein kinases, like the cellular protein tyrosine kinases, are predominantly intracellular.

Diabetes, angiogenesis, psoriasis, restenosis, ocular diseases, schizophrenia, rheumatoid arthritis, cardiovascular disease and cancer are exemplary of pathogenic conditions that have been linked with abnormal protein tyrosine kinase activity. Thus, a need exists for selective and potent small-molecule protein tyrosine kinase inhibitors. U.S. Pat. Nos. 6,383,790; 6,346,625; 6,235,746; 6,100,254 and PCT International Applications WO 01/47897, WO 00/27820 and WO 02/068406 are indicative of recent attempts to synthesize such inhibitors.

### SUMMARY OF THE INVENTION

The invention addresses the current need for selective and potent protein tyrosine kinase inhibitors by providing potent inhibitors of c-fms kinase. The invention is directed to the novel compounds of Formula 1: 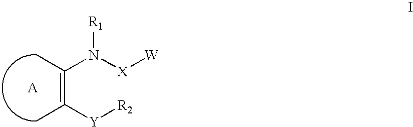
or a solvate, hydrate, tautomer or pharmaceutically acceptable salt thereof, wherein

A is
phenyl, naphthyl or biphenyl, each of which may be optionally substituted with one or more of -C₁₋₆ alkyl, amino, aminoalkyl, hydroxyalkyl, alkoxyalkyl, sulfonamidoalkyl, guanidinoalkyl, heteroaryl, halogen, hydroxy, —CF₃, alkoxy, aryl, aralkyl, heteroaralkyl, aryloxy, arylalkoxy, —OCF₃, —OCO-alkyl, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b}; or
a 5- to 7-membered mono- or a 8- to 10-membered bicyclic heteroaromatic ring having from one to four heteroatoms selected from N, O or S, and may be optionally substituted with one or more of -C₁₋₆ alkyl, amino, aminoalkyl, hydroxyalkyl, alkoxyalkyl, sulfonamidoalkyl, guanidinoalkyl, heteroaryl, halogen, hydroxy, —CF₃, alkoxy, aryl, aralkyl, heteroaralkyl, aryloxy, arylalkoxy, —OCF₃, —OCO-alkyl, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b};
   R₁ is
—H, aryl, —CORₐ, —CORₐ, —COORₐ, —CONRₐR_{b}, —SO₂Rₐ or —SO₂NRₐR_{b};
   X is
—CO—, —C(═NH)—, —CS—, —CON(Rₐ)—, —CS(NRₐ)—, —SO₂— or —CRₐR_{b}—;
   Y is
—S—, —SO—, —SO₂—, —O— or direct link;
   R₂ is
alkyl, cycloalkyl, heterocyclyl, aryl or heteroaryl, each of which may be optionally substituted with one or more halogens; and
   W is
phenyl, naphthyl or biphenyl, each of which may be optionally substituted with one or more of C₁₋₄ alkyl, amino, aminoalkyl, hydroxyalkyl, alkoxyalkyl, halogen, hydroxy, —CF₃, alkoxy, aryloxy, arylalkoxy, —OCF₃, —CORₐ, —CN, —C(NH)NH₂, —COORₐ, —CONRₐR_{b}, —NHCORₐR_{b}, —NHSO₂Rₐ, —NO₂, —SORₐ, —SO₃Rₐ or —SO₂NRₐR_{b}; or
a 5- to 6-membered mono- or a 8- to 10-membered bicyclic heterocyclic or heteroaromatic ring having from one to four heteroatoms selected from N, O or S, and may be optionally substituted with -C₁₋₆ alkyl, amino, aminoalkyl, hydroxyalkyl, alkoxyalkyl, heteroaryl, halogen, hydroxy, —CF₃, alkoxy, aryl, aralkyl, heteroaralkyl, aryloxy, arylalkoxy, —OCF₃, —OCO-alkyl, —OCO-alkylamino, —OCO-alkylamido, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b}.

The compounds of Formulae I are especially potent inhibitors of the c-fms protein tyrosine kinase.

The invention also relates to methods of inhibiting protein tyrosine kinase activity in a mammal by administration of a therapeutically effective amount of at least one compound of Formula I.

### DETAILED DESCRIPTION OF THE INVENTION

The invention is directed to the novel compounds of Formula I: 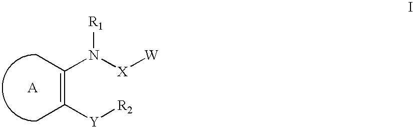
or a solvate, hydrate, tautomer or pharmaceutically acceptable salt thereof, wherein

A is
phenyl, naphthyl or biphenyl, each of which may be optionally substituted with one or more of -C₁₋₆ alkyl, amino, aminoalkyl, hydroxyalkyl, alkoxyalkyl, sulfonamidoalkyl, guanidinoalkyl, heteroaryl, halogen, hydroxy, —CF₃, alkoxy, aryl, aralkyl, heteroaralkyl, aryloxy, arylalkoxy, —OCF₃, —OCO-alkyl, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b}; or
a 5- to 7-membered mono- or a 8- to 10-membered bicyclic heteroaromatic ring having from one to four heteroatoms selected from N, O or S, and may be optionally substituted with one or more of -C₁₋₆ alkyl, amino, aminoalkyl, hydroxyalkyl, alkoxyalkyl, sulfonamidoalkyl, guanidinoalkyl, heteroaryl, halogen, hydroxy, —CF₃, alkoxy, aryl, aralkyl, heteroaralkyl, aryloxy, arylalkoxy, —OCF₃, —OCO-alkyl, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b};
   R₁ is
—H, aryl, —CORₐ, —CORₐ, —COORₐ, —CONRₐR_{b}, —SO₂Rₐ or —SO₂NRₐR_{b};
   X is
—CO—, —C(═NH)—, —CS—, —CON(Rₐ)—, —CS(NRₐ)—, —SO₂— or —CRₐR_{b}—;
   Y is
—S—, —SO—, —SO₂—, —O— or direct link;
   R₂ is
alkyl, cycloalkyl, heterocyclyl, aryl or heteroaryl, each of which may be optionally substituted with one or more halogens; and
   W is
phenyl, naphthyl or biphenyl, each of which may be optionally substituted with one or more of C₁₋₄ alkyl, amino, aminoalkyl, hydroxyalkyl, alkoxyalkyl, halogen, hydroxy, —CF₃, alkoxy, aryloxy, arylalkoxy, —OCF₃, —CORₐ, —CN, —C(NH)NH₂, —COORₐ, —CONRₐR_{b}, —NHCORₐR_{b}, —NHSO₂Rₐ, —NO₂, —SORₐ, —SO₃Rₐ or —SO₂NRₐR_{b}; or
a 5- to 6-membered mono- or a 8- to 10-membered bicyclic heterocyclic or heteroaromatic ring having from one to four heteroatoms selected from N, O or S, and may be optionally substituted with -C₁₋₆ alkyl, amino, aminoalkyl, hydroxyalkyl, alkoxyalkyl, heteroaryl, halogen, hydroxy, —CF₃, alkoxy, aryl, aralkyl, heteroaralkyl, aryloxy, arylalkoxy, —OCF₃, —OCO-alkyl, —OCO-alkylamino, —OCO-alkylamido, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b},
   wherein Rₐ and R_{b} are independently hydrogen, alkyl, cycloalkyl, haloalkyl, aryl, aralkyl, heteroaralkyl or heteroaryl.

A preferred subset of compounds is depicted in Formula II: 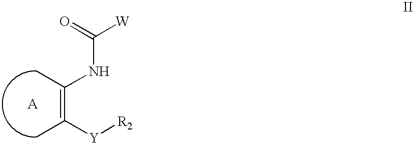
or a solvate, hydrate, tautomer or pharmaceutically acceptable salt thereof, wherein

A is
phenyl, which may be optionally substituted with one or more of -C₁₋₆ alkyl, amino, aminoalkyl, hydroxyalkyl, alkoxyalkyl, sulfonamidoalkyl, guanidinoalkyl, heteroaryl, halogen, hydroxy, —CF₃, alkoxy, aryl, aralkyl, heteroaralkyl, aryloxy, arylalkoxy, —OCF₃, —OCO-alkyl, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b};

Y is
a direct bond, —O—, or —S—;

R₂ is
alkyl, cycloalkyl, heterocyclyl, aryl or heteroaryl, each of which may be optionally substituted with one or more halogens; and

W is
furan, imidazole, or pyrrole each of which may be optionally substituted with -C₁₋₆ alkyl, amino, aminoalkyl, hydroxyalkyl, alkoxyalkyl, heteroaryl, halogen, hydroxy, —CF₃, alkoxy, aryl, aralkyl, heteroaralkyl, aryloxy, arylalkoxy, —OCF₃, —OCO-alkyl, —OCO-alkylamino, —OCO-alkylamido, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b},
   wherein Rₐ and R_{b} are independently hydrogen, alkyl, cycloalkyl, haloalkyl, aryl, aralkyl, heteroaralkyl or heteroaryl.

One group of preferred compounds of formula II are those wherein:

A is
phenyl;

Y is
a direct bond, —O—, or —S—;

R₂ is
alkyl, cycloalkyl, heterocyclyl, aryl or heteroaryl, each of which may be optionally substituted with one or more halogens; and

W is
furan which may be optionally substituted with —CN, or —NO₂.

One group of especially preferred compounds of Formula II are those wherein:

A is
phenyl;

Y is
a direct bond;

R₂ is
cycloalkyl, heterocyclyl, aryl or heteroaryl;

W is
cyanofuran;

Examples of especially preferred compounds of Formula II are:
5-Cyano-furan-2-carboxylic acid (2-cyclohex-1-enyl-phenyl)-amide,
5-Cyano-furan-2-carboxylic acid biphenyl-2-ylamide,
5-Cyano-furan-2-carboxylic acid [2-(5-methyl-furan-2-yl)-phenyl]-amide,
5-Cyano-furan-2-carboxylic acid (2-furan-2-yl-phenyl)-amide,
5-Cyano-furan-2-carboxylic acid (2-thiophen-2-yl-phenyl)-amide,
5-Cyano-furan-2-carboxylic acid (2-thiophen-3-yl-phenyl)-amide,
5-Cyano-furan-2-carboxylic acid (2-furan-3-yl-phenyl)-amide,
5-Cyano-furan-2-carboxylic acid (2-cyclohexyl-phenyl)-amide,
5-Cyano-furan-2-carboxylic acid [2-(1H-pyrazol-3-yl)-phenyl]-amide,
   and pharmaceutically acceptable salts thereof.

Another group of especially preferred compounds of Formula II are those wherein:

A is
phenyl;

Y is
—O— or —S—;

R₂ is
alkyl, which is optionally substituted with up to five halogens;

W is
nitrofuran;
   Additional examples of especially preferred compounds are:
5-nitro-furan-2-carboxylic acid [2-(2-chloro-1,1,2-trifluoro-ethylsulfanyl)-phenyl]-amide,
5-nitro-furan-2-carboxylic acid (2-ethoxyphenyl)-amide,
   and pharmaceutically acceptable salts thereof.

The invention also relates to methods of inhibiting protein tyrosine kinase activity in a mammal by administration of a therapeutically effective amount of at least one compound of Formula I. A preferred tyrosine kinase is c-fms.

The invention is considered to include the enantiomeric, diastereomeric and tautomeric forms of all compounds of Formulae I as well as their racemic mixtures. In addition, some of the compounds represented by Formulae I may be prodrugs, i.e., derivatives of an acting drug that possess superior delivery capabilities and therapeutic value as compared to the acting drug. Prodrugs are transformed into active drugs by in vivo enzymatic or chemical processes.

I. Definitions

The term “alkyl” refers to both linear and branched chain radicals of up to 12 carbon atoms, unless otherwise indicated, and includes, but is not limited to, methyl, ethyl, propyl, isopropyl, butyl, isobutyl, sec-butyl, tert-butyl, pentyl, isopentyl, hexyl, isohexyl, heptyl, octyl, 2,2,4-trimethylpentyl, nonyl, decyl, undecyl and dodecyl.

The term “cycloalkyl” refers to a saturated or partially unsaturated ring composed of from 3 to 8 carbon atoms. Alkyl substituents may optionally be present on the ring. Examples include cyclopropyl, 1,1-dimethyl cyclobutyl, 1,2,3-trimethylcyclopentyl, cyclohexyl and cyclohexenyl.

The term “heterocyclyl” refers to a nonaromatic (i.e. saturated or partially unsaturated) ring composed of from 3 to 7 carbon atoms and at least one heteroatom selected from N, O or S. Alkyl substituents may optionally be present on the ring. Examples include tetrahydrofuryl, dihydropyranyl, piperidyl, 2,5-dimethypiperidyl, morpholinyl, piperazinyl, thiomorpholinyl, pyrrolidinyl, pyrrolinyl, pyrazolidinyl, pyrazolinyl, imidazolidinyl and imidazolinyl.

The term “heterocyclylalkyl” refers to a C₁₋₆ alkyl group containing a heterocyclyl substituent. Examples include dihydropyranylethyl and 2-morpholinylpropyl.

The term “hydroxyalkyl” refers to at least one hydroxyl group bonded to any carbon atom along an alkyl chain.

The term “aminoalkyl” refers to at least one primary or secondary amino group bonded to any carbon atom along an alkyl chain; the term “aminoalkyl” may be used interchangeably with the term “alkylamino”.

The term “alkoxyalkyl” refers to at least one alkoxy group bonded to any carbon atom along an alkyl chain.

The term “polyalkoxyalkyl” refers to long-chain alkoxy compounds and includes polyethylene glycols of discreet or monodispersed sizes.

The term “thioalkyl” refers to at least one sulfur group bonded to any carbon atom along an alkyl chain. The sulfur group may be at any oxidation state and includes sulfoxides, sulfones and sulfates.

The term “carboxyalkyl” refers to at least one carboxylate group bonded to any carbon atom along an alkyl chain. The term “carboxylate group” includes carboxylic acids and alkyl, cycloalkyl, aryl or aralkyl carboxylate esters.

The term “heteroaromatic” or “heteroaryl” refers to 5- to 7-membered mono- or 8- to 10-membered bicyclic aromatic ring systems, any ring of which may contain from one to four heteroatoms selected from N, O or S where the nitrogen and sulfur atoms can exist in any allowed oxidation state. Examples include benzimidazolyl, benzothiazolyl, benzothienyl, benzoxazolyl, furyl, imidazolyl, isothiazolyl, isoxazolyl, oxazolyl, pyrazinyl, pyrazolyl, pyridyl, pyrimidinyl, pyrrolyl, quinolinyl, thiazolyl and thienyl.

The term “heteroaralkyl” refers to a C₁₋₆ alkyl group having a heteroaryl substituent. Examples include furylethyl and 2-quinolinylpropyl.

The term “heteroatom” refers to a nitrogen atom, an oxygen atom or a sulfur atom wherein the nitrogen and sulfur atoms can exist in any allowed oxidation states.

The term “alkoxy” refers to straight or branched chain radicals of up to 12 carbon atoms, unless otherwise indicated, bonded to an oxygen atom. Examples include methoxy, ethoxy, propoxy, isopropoxy and butoxy.

The term “aryl” refers to monocyclic or bicyclic aromatic ring systems containing from 6 to 12 carbons in the ring. Alkyl substituents may optionally be present on the ring. Examples include benzene, biphenyl and napththalene.

The term “aralkyl” refers to a C₁₋₆ alkyl group containing an aryl substituent. Examples include benzyl, phenylethyl or 2-naphthylmethyl.

The term “heteroaralkyl” refers to a C₁₋₆ alkyl group containing a heteroaryl substituent. Examples include furylmethyl and pyridylpropyl.

The term “aryloxy” refers to an oxygen atom bound to an aryl substituent. Examples include phenoxy and benzyloxy.

The term “arylalkoxy” refers to an alkoxy group bound to an aryl substituent. Examples include phenylmethyl ether.

The term “acyl” refers to the group —C(O)Rₐ, where Rₐ is alkyl, aryl, aralkyl, heteroaryl and heteroaralkyl. An “acylating agent” adds the —C(O)Rₐ group to a molecule.

The term “sulfonyl” refers to the group —S(O)₂Rₐ, where Rₐ is hydrogen, alkyl, cycloalkyl, haloalkyl, aryl, aralkyl, heteroaryl and heteroaralkyl. A “sulfonylating agent” adds the —S(O)₂Rₐ group to a molecule.

II. Therapeutic Uses

The compounds of Formula I represent novel potent inhibitors of protein tyrosine kinases, such as c-fms, and may be useful in the prevention and treatment of disorders resulting from actions of these kinases.

The invention also provides methods of inhibiting a protein tyrosine kinase comprising contacting the protein tyrosine kinase with an effective inhibitory amount of at least one of the compounds of Formula I. A preferred tyrosine kinase is c-fms. In one embodiment of inhibiting a protein tyrosine kinase, at least one of the compounds of Formula I is combined with a known tyrosine kinase inhibitor.

In various embodiments of the invention, the protein tyrosine kinases inhibited by the compounds of Formula I are located in cells, in a mammal or in vitro. In the case of mammals, which includes humans, a therapeutically effective amount of a pharmaceutically acceptable form of at least one of the compounds of Formula I is administered.

The invention further provides methods of treating cancer in mammals, including humans, by administration of a therapeutically effective amount of a pharmaceutically acceptable composition of least one compound of Formula I. Exemplary cancers include, but are not limited to, ovarian cancer, uterine cancer, breast cancer, colon cancer, stomach cancer, hairy cell leukemia and non-small lung carcinoma. In one embodiment of the invention, an effective amount of at least one compound of Formula I is administered in combination with an effective amount of a chemotherapeutic agent.

The invention also provides methods of treating cardiovascular and inflammatory diseases in mammals, including humans, by administration of a therapeutically effective amount of a pharmaceutically acceptable form of at least one of the compounds of Formula I. Examples of diseases that may be effectively treated include atherosclerosis, cardiac hypertrophy, glomerulonephritis, rheumatoid arthritis, psoriasis, diabetes, tumor related angiogenesis, restenosis, schizophrenia and Alzheimer's dementia.

When employed as protein tyrosine kinase inhibitors, the compounds of the invention may be administered in an effective amount within the dosage range of about 0.5 mg to about 10 g, preferably between about 0.5 mg to about 5 g, in single or divided daily doses. The dosage administered will be affected by factors such as the route of administration, the health, weight and age of the recipient, the frequency of the treatment and the presence of concurrent and unrelated treatments.

The compounds of Formula I may be formulated into pharmaceutical compositions comprising any known pharmaceutically acceptable carriers. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

The pharmaceutically-acceptable salts of the compounds of Formula I include the conventional non-toxic salts or the quaternary ammonium salts which are formed from inorganic or organic acids or bases. Examples of such acid addition salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Base salts include ammonium salts, alkali metal salts such as sodium and potassium salts, alkaline earth metal salts such as calcium and magnesium salts, salts with organic bases such as dicyclohexylamine salts and salts with amino acids such as arginine. Also, the basic nitrogen-containing groups may be quaternized with, for example, alkyl halides.

The pharmaceutical compositions of the invention may be administered by any means that accomplish their intended purpose. Examples include administration by parenteral, subcutaneous, intravenous, intramuscular, intraperitoneal, transdermal, buccal or ocular routes. Alternatively or concurrently, administration may be by the oral route. Suitable formulations for parenteral administration include aqueous solutions of the active compounds in water-soluble form, for example, water-soluble salts, acidic solutions, alkaline solutions, dextrose-water solutions, isotonic carbohydrate solutions and cyclodextrin inclusion complexes.
III. Methods of Preparation

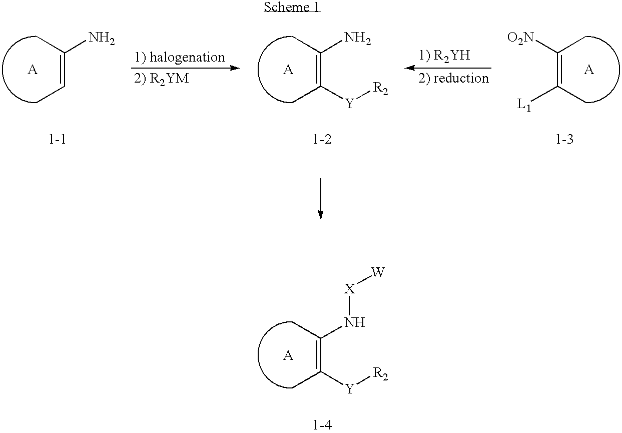

Scheme 1 illustrates general methodology for the preparation of compounds of Formula I.

Compounds of Formula 1-2 where Y is a direct link can be obtained by ortho-halogenation, preferably bromination, of amino compounds of Formula 1-1 followed by metal-catalyzed coupling reactions with boronic acids or boronate esters (Suzuki reactions, where R₂YM is R₂B(OH)₂ or a boronic ester) or tin reagents (Stille reactions, where R₂YM is R₂Sn(alkyl)₃) (for reviews, see N. Miyaura, A. Suzuki, Chem. Rev., 95:2457 (1995), J. K. Stille, Angew. Chem, Int. Ed. Engl., 25: 508024 (1986) and A. Suzuki in Metal-Catalyzed Coupling Reactions, F. Deiderich, P. Stang, Eds., Wiley-VCH, Weinheim (1988)). The preferred conditions for bromination are N-bromosuccinimide (NBS) in a suitable solvent such as N,N-dimethylformamide (DMF), dichloromethane (DCM) or acetonitrile. Metal-catalyzed couplings, preferably Suzuki reactions, can be performed according to standard methodology, preferably in the presence of a palladium catalyst such as tetrakis(triphenylphosphine)palladium(0) (Pd(PPh₃)₄), an aqueous base such aq. Na₂CO₃, and a suitable solvent such as toluene, ethanol, dimethoxyethane (DME), or DMF.

Compounds of Formula 1-2 where Y is —O— or —S— can be obtained by nucleophilic aromatic substitution on compounds of Formula 1-3 (where L₁ is a leaving group such as a halogen, preferably fluoro or chloro) with alcohols R₂OH to form ethers (Y=—O—) or with thiols R₂SH to form sulfides (Y=—S—) followed by reduction of the nitro group. Nucleophilic aromatic displacements can be performed in the presence of a suitable base such as triethylamine (NEt₃) or K₂CO₃ in a suitable solvent such as DMF. Nitro reductions can be performed according to standard synthetic methodologies (for a review, see M. Hudlicky, Reduction in Organic Chemistry, Wiley, NY (1984)) and include preferred methods such as palladium-catalyzed hydrogenolysis or treatment with iron (0) and NH₄Cl (see, for example, S. Mitsumori, et al, J. Med. Chem., 46: 2436-45 (2003)).

Compounds of Formula 1-2 where Y is —SO—, or —SO₂—, can be obtained from compounds of Formula 1-3 by nucleophilic aromatic displacement with thiols as described above followed by sulfur oxidation and nitro group reduction. Sulfoxides can be obtained by oxidation using an appropriate oxidant such as one equivalent of MCPBA or by treatment with NaIO₄ (see, for example, J. Regan, et al, J. Med. Chem., 46: 4676-86 (2003)) and sulfones can be obtained using two equivalents of MCPBA or by treatment with 4-methylmorpholine N-oxide and catalytic osmium tetroxide (see, for example, PCT application WO 01/47919). Alternatively, compounds of Formula 1-2 where Y is —SO₂— may be obtained by nucleophilic aromatic substitution of nitrohalo compounds with sulfinate anions WSO₂M (M=Li, Na or K; see, for example, L. Field and R. D. Clark, Organic Synthesis, Collective Vol. IV: 674-677, John Wiley and Sons, Inc. (1963)) followed by nitro group reduction as described above.

Compounds of Formula 1-4 where X is —CO— can be prepared by reaction of compounds of Formula 1-2 with carboxylic acids WCOOH according to standard procedures for amide bond formation (for a review, see: M. Bodansky and A. Bodansky, The Practice of Peptide Synthesis, Springer-Verlag, NY (1984)) or by reaction with acid chlorides WCOCl or activated esters WCO₂Rq (where Rq is a leaving group such as pentafluorophenyl or N-succinimide). The preferred reaction conditions for coupling with WCOOH are: when W is a furan, oxalyl chloride in DCM with DMF as a catalyst to form the acid chloride WCOCl and then coupling in the presence of a trialkylamine such as DIEA; when W is a pyrrole, 1-(3-dimethylaminopropyl)-3-ethylcarbodiimide hydrochloride (EDCI) and 1-hydroxybenzotriazole-6-sulfonamidomethyl hydrochloride (HOBt); and when W is an imidazole, the preferred conditions are bromotripyrrolidinophosphonium hexafluorophosphate (PyBrOP) and diisopropylethylamine (DIEA) in DCM.

Compounds of Formula of 1-4 where X is —SO₂— can be prepared by reaction of sulfonyl chlorides WSO₂Cl or sulfonyl bromides WSO₂Br with compounds of Formula 1-2 in the presence of a suitable base such as NEt₃.

Compounds of Formula 1-4 where X is —C(═NH)— can be obtained by reaction of compounds of Formula 1-2 with imidates WC(C═NH)ORs (where Rs is C₁-C₄ alkyl, preferably methyl or ethyl) in the presence of a base such as NEt₃. Alternatively, compounds of Formula 1-4 may be formed by reaction of compounds of Formula 1-2 with nitriles WCN in the presence of a Lewis acid such as trimethylaluminum (see R. Garigipati, Tetrahedron Lett., 31:1969-71 (1990)), copper (I) chloride (see G. Rousselet, et al, Tetrahedron Lett., 34:6395 (1993)), or Ln (III) salts (see J. Fosberg, et al, J. Org. Chem., 52: 1017 (1987). Finally, these compounds can be obtained by coupling of compounds of Formula 1-2 with acids WCO₂H by the procedures outlined above when X is —CO— to form the amides followed by treatment with NH₄Cl/Me₃Al as described above by Garigipati, idem.

Compounds of Formula 1-4 where X is —CON(Rₐ)— can be obtained by reaction of compounds of Formula 1-2 with isocyanates WNCO where Rₐ is H or with carbamyl chlorides WN(Rₐ)COCl (where Rₐ is not H) in the presence of an acid scavenger such as a trialkylamine (for example DIEA) according to standard synthetic procedures for urea formation.

Compounds of Formula 1-4 where X is —CRₐR_{b}— can be obtaining by reaction of compounds of Formula 1-4 with aldehydes WCHO (where Rₐ and R_{b} are H) or ketones WCORₐ (where R_{b} is H) in the presence of a suitable reducing agent such as sodium triacetoxyborohydride (for examples, see A. F Abdel-Magid, et al, J. Org. Chem., 61: 3849-62 (1996)). Alternatively, reaction of compounds of Formula WCRₐ-L₂ (where L₂ is an appropriate leaving group such —Br, —I, —Cl, -OMs, or -OTs) with compounds of Formula 1-2 in the presence of a base such as K₂CO₃ or NEt₃ can provide compounds of Formula 1-4.

Compounds of Formula 1-4 when X is —CS—, or —CS(NRₐ)— can be obtained by treatment of compounds of Formula 1-4 where X is —CO—, or —CON(Rₐ)—, respectively, with P₂S₅ (see, for example, J. S. Davidson, Synthesis., 359-61 (1979)) or Lawesson's reagent (S.-O., Lawesson, et al. Bull. Soc. Chim. Belg. 87, 223, 293 (1978); for a review see M. P. Cava and M. I. Levinson, Tetrahedron, 41: 5061 (1985).

It is understood that the optional substitution present on ring A in Formula I may be present in the starting materials 1-1 or 1-3 and, in such cases, would be carried through the synthesis outlined in Scheme 1. Alternatively various substituents on compounds of Formula I may be introduced in a number of ways described below to provide the optional substitution listed for Formula I and, in particular, can also be further used to produce compounds of Formulae II, III and IV. Leaving groups present on ring A in Formula 1-1 or 1-3, can be substituted before or at any step during Scheme 1. When such leaving groups (preferably fluoro or chloro) are activated by the nitro group of Formula 1-3 for nucleophilic attack, they can undergo direct nucleophilic aromatic substitution by ammonia and azide anion or by amines, alcohols, thiols and other nucleophiles in the presence of a suitable base such as K₂CO₃, N,N-diisopropylethylamine (DIEA) or NEt₃. When the leaving group is suitable for metal-catalyzed couplings (preferably bromo or trifluoromethanesulfonyloxy), a number of cross-coupling reactions (such as Suzuki or Stille reactions as discussed above for the introduction of R₂Y where Y is a direct bond) may be performed. Other metal-catalyzed coupling reactions that can be employed include aromatic and heteroaromatic amination and amidation (for reviews, see: S. L. Buchwald, et al, Top. Curr. Chem., 219:131-209 (2001) and J. F. Hartwig in “Organopalladium Chemistry for Organic Synthesis,” Wiley Interscience, NY (2002).

In some cases, the substituents of Formula 1-4 can be further derivatized as described below to provide diverse examples of Formula I. Protecting groups on compounds of Formula 1-4 can be removed according to standard synthetic methodologies (Theodora W. Greene and Peter G. M. Wuts, John Wiley and Sons, Inc., NY (1991)) and can be then subjected to further derivatization. Examples of further derivatization of compounds of 1-4 to provide compounds of Formulae 1-5 include, but are not limited to: when compounds of Formula 1-4 contain a primary or secondary amine, the amine may be reacted with aldehydes or ketones in the presence of a reducing agent such as sodium triacetoxyborohydride (see Abdel-Magid reference above) to reductively alkylate; with acid chlorides or carboxylic acids and an amide bond forming reagent as described above to form amides; with sulfonyl chlorides to form sulfonamides; with isocyanates to form ureas; with aryl- or heteroaryl-halides in the presence of a palladium catalyst as described above (see Buchwald and Hartwig references above) to form aryl and heteroarylamines. In addition, when compounds of Formulae 1-4 contain an aryl halide or heteroaryl halide, these compounds may be subjected to metal-catalyzed reactions with boronic acids (for example, Suzuki or Stille couplings as described above), or, amines or alcohols (Buchwald- or Hartwig-type couplings, see Buchwald and Hartwig references above). When compounds of Formulae 1-4 contain a cyano group, this group may be hydrolyzed to amides or acids under acid or basic conditions. Basic amines may be oxidized to N-oxides and conversely N-oxides may be reduced to basic amines. When compounds of Formula 1-4 contain a sulfide, either acyclic or cyclic (as in the case of compounds of Formula III), the sulfide can be further oxidized to the corresponding sulfoxides or sulfones (as previously described above for compounds where Y is —SO—, or —SO₂—).

### EXAMPLE 1

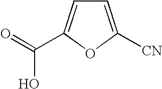

To a flask with a stir bar and Vigreaux column under Ar was added 2-formyl-5-furancarboxylic acid (2.8 g, 20 mmol), hydroxylamine hydrochloride (2.7 g, 40 mmol), and dry pyridine (50 mL). The mixture was heated to 85° C., acetic anhydride (40 mL) was added and the mixture was stirred for 3 h. After cooling to 60° C., water (250 mL) was added and the mixture was stirred at room temperature (RT) for 70 h. The mixture was acidified to pH 2 with concentrated hydrochloric acid and extracted with 3:1 dichloromethane-isopropanol (8×100 mL). The combined organic layers were washed with water (100 mL), brine (100 mL), dried over and sodium sulfate and concentrated in vacuo to afford the title compound as a tan solid (1.26 g, 46%). ¹H-NMR (CD₃OD; 400 MHz): δ 14.05 (br s, 1H), 7.74 (d, 1H, J=3.8 Hz), 7.42 (d, 1H, J=3.8 Hz).

### EXAMPLE 2

### 5-Cyano-furan-2-carboxylic acid (2-furan-2-yl-phenyl)-amide

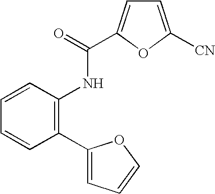 a) 2-Furan-2-yl-aniline

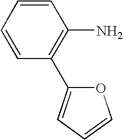

To a mixture of 2-bromoaniline (100 mg, 0.58 mmol) and 2-furanboronic acid (65 mg, 0.58 mmol) under Ar was added degassed 2M aq Na₂CO₃ (3.0 mL, 6.0 mmol), Pd(PPh₃)₄ (34 mg, 0.030 mmol), ethanol (3 mL) and toluene (6 mL). The mixture was stirred for 3 h at 80° C. After cooling to RT, ethyl acetate (EtOAc) (20 mL) and saturated aq NaHCO₃ (20 mL) were added and the organic layer was concentrated in vacuo. The residue was concentrated from dichloromethane and methanol, then purified by preparative TLC on silica gel (10% ethyl acetate in hexane) to afford the title compound as a light brown oil (45 mg, 51%). ¹H-NMR (CDCl₃; 400 MHz): δ 7.50-6.50 (m, 7H), 4.23 (br s, 2H).
b) 5-Cyano-furan-2-carboxylic acid (2-furan-2-yl-phenyl)-amide

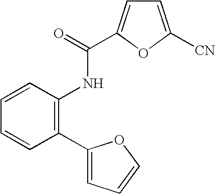

To 2-cyano-5-furancarboxylic acid (as prepared in Example 1, 41 mg, 0.30 mmol), under argon was added dichloromethane (10 mL), N,N-dimethylformamide (DMF) (100 μL), and oxalyl chloride (29 μL, 0.33 mmol) and the mixture stirred for 35 min. The solvents were removed in vacuo and the residue concentrated from toluene (2×5 mL), taken up in DMF (4 mL), and added to a solution of 2-furan-2-ylaniline (as prepared in the previous step, 50 mg, 0.3 mmol) and N,N-diisopropylethylamine (DIEA) (157 μL, 0.901 mmol) in DMF (2 mL). After 3.25 h, a second equivalent portion of acid chloride (0.30 mmol, prepared as above) was added and the mixture was stirred at RT for 67 h. The mixture was poured into saturated sodium bicarbonate (50 mL), and extracted with dichloromethane (3×20 mL). The organic layers were washed with water (50 mL), brine (50 mL), dried over anhydrous sodium sulfate and concentrated in vacuo. Purification of the residue by silica gel preparative thin layer chromatography (TLC) eluting with 30% ethyl acetate in hexanes yielded the title compound as a light tan solid (9.5 mg, 11%). ¹H-NMR (CDCl₃; 400 MHz): δ 9.90 (br s, 1H), 8.49 (dd, 1H, J=7.4, 1.1 Hz), 7.68 (dd, 1H, J=1.8, 0.7 Hz), 7.38 (m, 1H), 7.30 (d, 1H, J=3.7 Hz), 7.23 (d, 1H, J=3.7 Hz), 7.20 (dd, 1H, J=7.5, 1.2 Hz), 6.71 (dd, 1H, J=3.4, 0.7 Hz), 6.61 (m, 1H). LC-MS (ESI, m/z): Calcd. for C₁₆H₁₁N₂O₃: 279.1 (M+H); found: 279.1.

### EXAMPLE 3

### 5-Cyano-furan-2-carboxylic acid (2-furan-3-yl-phenyl)-amide

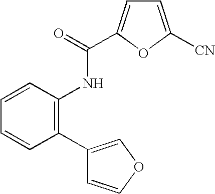 a) 2-Furan-3-yl-aniline

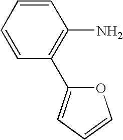

To a mixture of 2-bromoaniline (200 mg, 1.16 mmol) and 3-furanboronic acid (130 mg, 1.16 mmol) under argon was added 2M aq Na₂CO₃ (6.0 mL, 12 mmol), Pd(PPh₃)₄ (68 mg, 0.06 mmol), ethanol (6 mL) and toluene (12 mL). The mixture was stirred for 3 h at 80° C. After cooling to RT, EtOAc (50 mL) and saturated aq NaHCO₃ (50 mL) were added and the layers were separated. The organic layer was concentrated in vacuo and the resulting residue was concentrated from dichloromethane and methanol and then purified by preparative TLC on silica gel (10% ethyl acetate in hexane), which afforded the title compound as a light brown oil (109 mg, 59%). ¹H-NMR (CDCl₃; 400 MHz): δ 7.66 (dd, 1H, J=1.9, 1.6 Hz), 7.52 (t, 1H, J=1.7 Hz), 7.21 (dd, 1H, J=7.6, 1.6 Hz), 7.13 (m, 1H), 6.82-6.80 (m, 1H), 6.78-6.76 (m, 1H), 7.64 (dd, 1H, J=1.8, 0.8 Hz), 3.85 (br s, 2H).
b) 5-Cyano-furan-2-carboxylic acid (2-furan-3-yl-phenyl)-amide

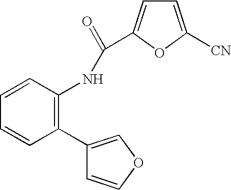

To 2-cyano-5-furancarboxylic acid (as prepared in Example 1, 93 mg, 0.68 mmol) under Ar was added dichloromethane (20 mL), DMF (50 μL), and oxalyl chloride (65 μL, 0.75 mmol) and the mixture stirred for 25 min. The solvents were removed in vacuo, and the residual brown solid was taken up in DMF (4 mL) and added to a solution of 2-(3-furyl)-aniline (as prepared in the previous step, 109 mg, 0.68 mmol) and DIEA (355 μL, 2 mmol) in DMF (8 mL). The mixture was stirred for 2 h 30 min at RT, then at 60° C. for 17 h. The mixture was poured into saturated aq sodium bicarbonate (100 mL), and extracted with dichloromethane (3×30 mL). The organic layers were washed with water (50 mL), brine (50 mL), dried over anhydrous sodium sulfate and concentrated in vacuo. Purification of the crude residue by silica gel preparative TLC eluting with 30% ethyl acetate in hexanes yielded the title compound as a light tan solid (23 mg, 12%). ¹H-NMR (CDCl₃; 400 MHz): δ 8.41 (br s, 1H), 8.36 (d, 1H, J=8.4 Hz), 7.66-7.64 (m, 2H), 7.42-7.36 (m, 2H), 7.29-7.20 (m, 3H), 7.23 (d, 1H, J=3.7 Hz), 7.20 (dd, 1H, J=7.5, 1.2 Hz), 6.71 (dd, 1H, J=3.4, 0.7 Hz), 6.65 (m, 1H). LC-MS (ESI, m/z): Calcd. for C₁₆H₁₁N₂O₃: 279.1 (M+H); found: 279.1.

### EXAMPLE 4

### 5-Cyano-furan-2-carboxylic acid biphenyl-2-ylamide

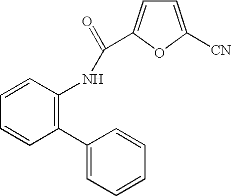

To 2-cyano-5-furancarboxylic acid (as prepared in Example 1, 100 mg, 0.73 mmol) under argon was added dichloromethane (20 mL), DMF (50 μL), and oxalyl chloride (70 μL, 0.8 mmol) and the mixture stirred for 1 h. The solvents were removed in vacuo and the residual dark yellow oil was taken up in dichloromethane (10 mL), added to a solution of 2-aminobiphenyl (56 mg, 0.33 mmol) and DIEA (173 μL, 0.993 mmol) in dichloromethane (5 mL). The mixture was stirred for 17 h at RT, poured into saturated aq sodium bicarbonate (50 mL), and extracted with dichloromethane (3×10 mL). The organic layers were washed with water (50 mL), brine (50 mL), dried over anhydrous sodium sulfate and concentrated in vacuo. Purification of the resulting residue by silica gel preparative TLC eluting with 30% ethyl acetate in hexanes yielded the title compound as an off-white solid (24 mg, 11%). ¹H-NMR (CDCl₃; 400 MHz): δ 8.44 (dd, 1H, J=8.2, 0.9 Hz), 8.32 (br s, 1H), 7.57-7.24 (m, 9H), 7.42-7.36 (m, 2H), 7.21 (d, 1H, J=3.7 Hz), 7.14 (d, 1H, J=3.7 Hz). LC-MS (ESI, m/z): Calcd. for C₁₈H₁₃N₂O₂: 289.1 (M+H); found: 288.9.

### EXAMPLE 5

### 5-Cyano-furan-2-carboxylic acid [2-(1 H-pyrazol-3-yl)-phenyl]-amide

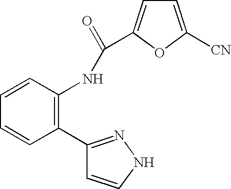 a) 2-(1H-Pyrazol-3-yl)-phenylamine

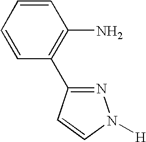

To 3-(2-nitrophenyl)-1H-pyrazole (Butt Park Ltd.) (100 mg, 0.53 mmol) was added methanol (10 mL) and the solution was degassed under Ar. To this solution was added 10% palladium on carbon (100 mg) and the mixture stirred at RT for 50 min under hydrogen (1 atm). The mixture was filtered through a short column of Celite, the product was washed off the column with methanol, and the solvent removed in vacuo to yield the title compound as a colorless crystalline solid (84 mg, quantitative yield). ¹H-NMR (CDCl₃; 400 MHz): δ 7.55 (d, 1H, J=2.3 Hz), 7.51 (m, 1H), 7.14 (m, 1H), 7.79-6.75 (m, 2H), 6.62 (m, 1H, J=2.4). LC-MS (ESI, m/z): Calcd. for C₉H₁₀N₃: 160.1 (M+H); found: 160.1.

b) 5-Cyano-furan-2-carboxylic acid [2-(1H-pyrazol-3-yl)-phenyl]-amide

To 2-cyano-5-furancarboxylic acid (as prepared in Example 1, 69 mg, 0.5 mmol) under Ar was added dichloromethane (10 mL), DMF (50 μL), and oxalyl chloride (48 μL, 0.55 mmol) and the mixture stirred for 1 h. The solvents were removed in vacuo, the residue was concentrated from toluene (5 mL), and then taken up in dichloromethane (5 mL). This solution was added to a stirred solution of 2-(1H-pyrazol-3-yl)-aniline (as prepared in the previous step) (80 mg, 0.5 mmol) and DIEA (261 μL, 1.5 mmol) in dichloromethane (5 mL). The mixture was stirred for 2.5 h at RT, poured into saturated aq sodium bicarbonate (50 mL), and extracted with dichloromethane (3×30 mL). The organic layers were washed with water (20 mL), brine (20 mL), dried over anhydrous sodium sulfate and concentrated in vacuo. Purification of the resulting residue by silica gel preparative TLC eluting with 10% methanol in dichloromethane yielded the title compound as an off-white solid (39 mg, 28%). ¹H-NMR (CDCl₃; 400 MHz): δ 8.41 (br s, 1H), 8.36 (d, 1H, J=8.4 Hz), 7.66-7.64 (m, 2H), 7.42-7.36 (m, 2H), 7.29-7.20 (m, 3H), 7.23 (d, 1H, J=3.7 Hz), 7.20 (dd, 1H, J=7.5, 1.2 Hz), 6.71 (dd, 1H, J=3.4, 0.7 Hz), 6.65 (m, 1H). LC-MS (ESI, m/z): Calcd. for C₁₅H₁₁N₄O₂: 279.1 (M+H); found: 279.0.

### EXAMPLE 6

### 5-Cyano-furan-2-carboxylic acid (2-cyclohex-1-enyl-phenyl)-amide

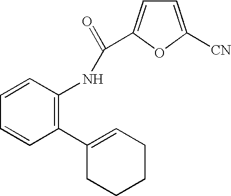 a) 2-Cyclohex-1-enyl-aniline

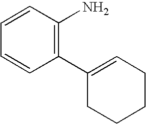

To a mixture of 2-bromoaniline (136 mg, 0.79 mmol), cyclohexene-1-yl-boronic acid (100 mg, 0.79 mmol) under argon was added 2M aq sodium carbonate (4.0 mL, 8.0 mmol), tetrakistriphenylphosphine palladium (0) (46 mg, 0.04 mmol), toluene (8 mL), and ethanol (4 mL) and the mixture was stirred at 80 C. for 4 h. The mixture was cooled to RT, poured into ethyl acetate (40 mL), washed with saturated aq sodium bicarbonate (40 mL) and the solvents were removed in vacuo. Purification of the resulting solid by silica gel preparative TLC eluting with acetonitrile, hexane, dichloromethane (10:50:40), and again with acetonitrile, hexane, dichloromethane (5:30:70) yielded the title compound as a colorless glass (30 mg, 22%). ¹H-NMR (CDCl₃; 400 MHz): δ 7.05-6.96 (m, 2H), 6.74-6.68 (m, 2H), 5.76 (m, 1H), 3.76 (br s, 2H), 3.21 (m, 4H), 2.40-2.14 (m, 4H), 1.80-1.66 (m, 4H). LC-MS (ESI, m/z): Calcd. for C₁₂H₁₆N: 174.1 (M+H); found: 174.1.

b) 5-Cyano-furan-2-carboxylic acid (2-cyclohex-1-enyl-phenyl)-amide

The title compound was prepared according to the procedure from Example 5, step (b) using 2-cyano-furan-2-carboxylic acid (as prepared in Example 1, 56 mg, 0.22 mmol) and 2-cyclohex-1-enyl-aniline (as prepared in the previous step, 30 mg, 0.4 mmol). Purification of the resulting residue by silica gel preparative TLC eluting with 10% methanol in dichloromethane yielded the title compound as an off-white solid (19 mg, 38%). ¹H-NMR (CDCl₃; 400 MHz): δ 8.82 (br s, 1H), 8.42 (d, 1H, J=7.8, 0.8 Hz), 7.32-7.12 (m, 5H), 5.88 (m, 1H), 2.32-2.23 (m, 4H), 1.84 (m, 4H). LC-MS (ESI, m/z): Calcd. for C₁₈H₁₇N₂O₂: 293.1 (M+H); found: 293.1.

### EXAMPLE 7

### 5-Cyano-furan-2-carboxylic acid (2-cyclohexylphenyl)-amide

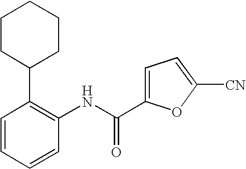 a) (2-Cyclohexylphenyl)-carbamic acid tert-butyl ester

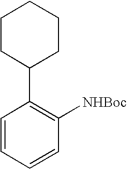

To a solution of 335 mg (1.64 mmol) of 2-cyclohexylbenzoic acid in 3.5 mL of anhydrous t-butanol was added 457 μL (3.28 mmol) of triethylamine followed by 388 μL (1.80 mmol) of diphenylphosphorylazide. After stirring for 5 min, the mixture was heated at 80° C. for 3 h. The mixture was then cooled to RT and concentrated to give a colorless oil that crystallized on standing. Chromatography on a 5-g silica solid-phase extraction (SPE) column with dichloromethane afforded 330 mg (73%) of the title compound as a colorless crystalline solid. Mass spectrum (ESI, m/z): Calcd. for C₁₇H₂₅NO₂, 176.1 (M−Boc+2H), found 176.6.
b) 2-Cyclohexylaniline trifluoroacetic acid salt

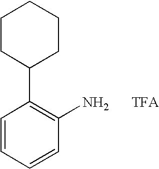

To 89.1 mg (0.324 mmol) of 2-(cyclohexylphenyl)-carbamic acid tert-butyl ester (as prepared in the previous step) was added 3 mL of a mixture of trifluoroacetic acid:dichloromethane:water (12:12:1 v/v/v) and the solution stirred at RT for 1.5 h. The solution was concentrated to afford 130 mg (73%) of the title compound as a colorless crystalline solid. Mass spectrum (ESI, m/z): Calcd. for C₁₂₇H₁₇N, 176.1 (M+H), found 176.2.
c) 5-Cyano-furan-2-carboxylic acid (2-cyclohexylphenyl)-amide

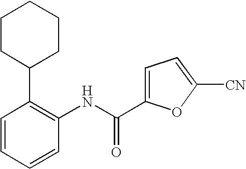

The title compound was prepared following the procedure of Example 5, step (b) using 25.5 mg (0.186 mmol) of 5-cyanofuran-2-carboxylic acid (as prepared in Example 1), 32.4 μL (0.372 mmol) of oxalyl chloride, 64.6 mg (0.223 mmol) of 2-cyclohexyl-aniline trifluoroacetic acid salt, and 64.8 μL (0.197 mmol) of DIEA. The resulting residue was chromatographed on a 5-g silica SPE column with 40-80% dichloromethane-hexane to afford 45.8 mg (84%) of the title compound as a white solid. ¹H-NMR (CDCl₃; 400 MHz) δ 8.06 (br s, 1H), 7.88 (m, 1H), 7.34 (m, 1H), 7.2-7.3 (m, 3H partially obscured by CHCl₃ peak), 2.66 (m, 1H), 1.82-1.94 (m, 5H), and 1.29-1.53 (m, 5H). Mass spectrum (ESI, m/z): Calcd. for C₁₈₁H₁₈N₂O₂, 295.1 (M+H), found 294.9.

IV. Results

An autophosphorylation, fluorescence polarization competition immunoassay was used to determine the potency for c-fms inhibition exhibited by selected compounds of Formula I. The assay was performed in black 96-well microplates (LJL BioSystems). The assay buffer used was 100 mM 4-(2-hydroxyethyl)piperazine 1-ethanesulfonic acid (HEPES), pH 7.5, 1 mM 1,4-dithio-DL-threitol (DTT), 0.01% (v/v) Tween-20. Compounds were diluted in assay buffer containing 4% dimethylsulfoxide (DMSO) just prior to the assay. To each well, 5 μL of compound were added followed by the addition of 3 μL of a mix containing 33 nM c-fms (Johnson & Johnson PRD) and 16.7 mM MgCl₂ (Sigma) in assay buffer. The kinase reaction was initiated by adding 2 μL of 5 mM ATP (Sigma) in assay buffer. The final concentrations in the assay were 10 nM c-fms, 1 mM ATP, 5 mM MgCl₂, 2% DMSO. Control reactions were ran in each plate: in positive and negative control wells, assay buffer (made 4% in DMSO) was substituted for the compound; in addition, positive control wells received 1.2 μL of 50 mM ethylenediaminetetraaceticacid (EDTA).

The plates were incubated at room temperature for 45 min. At the end of the incubation, the reaction was quenched with 1.2 μL of 50 mM EDTA (EDTA was not added to the positive control wells at this point; see above). Following a 5-min incubation, each well received 10 μL of a 1:1:3 mixture of anti-phosphotyrosine antibody, 10×, PTK green tracer, 10× (vortexed), FP dilution buffer, respectively (all from PanVera, cat. # P2837). The plate was covered, incubated for 30 min at room temperature and the fluorescence polarization was read on the Analyst. The instrument settings were: 485 nm excitation filter; 530 nm emission filter; Z height: middle of well; G factor: 0.93. Under these conditions, the fluorescence polarization values for positive and negative controls were approximately 300 and 150, respectively, and were used to define the 100% and 0% inhibition of the c-fms reaction. The reported IC₅₀ values are averages of three independent measurements.

Table 1 lists representative compounds of Formulae I and II of the invention.

TABLE 1

| | | % |
|---|---|---|
| | | Inhibition |
| Structure | IC_50 | at 2 μM |
| 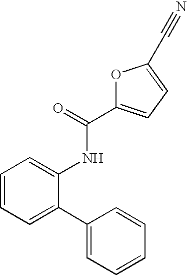 | <5 μM | 54 |
| 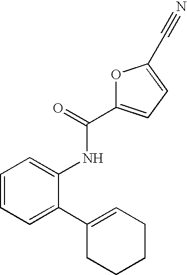 | <1 μM | 85 |
| 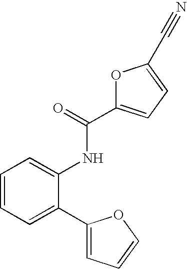 | ND | 13 |
| 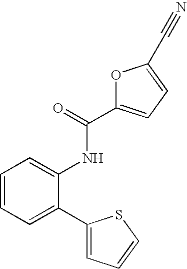 | ND | 25 |
| 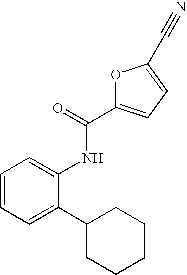 | ND | 29 |

ND: not determined

1. A compound of Formula I: 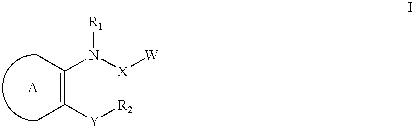
or a solvate, hydrate, tautomer or pharmaceutically acceptable salt thereof, wherein
A is
phenyl, naphthyl or biphenyl, each of which may be optionally substituted with one or more of -C₁₋₆ alkyl, amino, aminoalkyl, hydroxyalkyl, alkoxyalkyl, sulfonamidoalkyl, guanidinoalkyl, heteroaryl, halogen, hydroxy, —CF₃, alkoxy, aryl, aralkyl, heteroaralkyl, aryloxy, arylalkoxy, —OCF₃, —OCO-alkyl, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b}; or
a 5- to 7-membered mono- or a 8- to 10-membered bicyclic heteroaromatic ring having from one to four heteroatoms selected from N, O or S, and may be optionally substituted with one or more of -C₁₋₆ alkyl, amino, aminoalkyl, hydroxyalkyl, alkoxyalkyl, sulfonamidoalkyl, guanidinoalkyl, heteroaryl, halogen, hydroxy, —CF₃, alkoxy, aryl, aralkyl, heteroaralkyl, aryloxy, arylalkoxy, —OCF₃, —OCO-alkyl, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b};
R₁ is
—H, aryl, —CORₐ, —CORₐ, —COORₐ, —CONRₐR_{b}, —SO₂Rₐ or —SO₂NRₐR_{b};
X is
—CO—, —C(═NH)—, —CS—, —CON(Rₐ)—, —CS(NRₐ)—, —SO₂— or —CRₐR_{b}—;
Y is
—S—, —SO—, —SO₂—, —O— or direct link;
R₂ is
alkyl, cycloalkyl, heterocyclyl, aryl or heteroaryl, each of which may be optionally substituted with one or more halogens; and
W is
phenyl, naphthyl or biphenyl, each of which may be optionally substituted with one or more of C₁₋₄ alkyl, amino, aminoalkyl, hydroxyalkyl, alkoxyalkyl, halogen, hydroxy, —CF₃, alkoxy, aryloxy, arylalkoxy, —OCF₃, —CORₐ, —CN, —C(NH)NH₂, —COORₐ, —CONRₐR_{b}, —NHCORₐR_{b}, —NHSO₂Rₐ, —NO₂, —SORₐ, —SO₃Rₐ or —SO₂NRₐR_{b}; or
a 5- to 6-membered mono- or a 8- to 10-membered bicyclic heterocyclic or heteroaromatic ring having from one to four heteroatoms selected from N, O or S, and may be optionally substituted with -C₁₋₆ alkyl, amino, aminoalkyl, hydroxyalkyl, alkoxyalkyl, heteroaryl, halogen, hydroxy, —CF₃, alkoxy, aryl, aralkyl, heteroaralkyl, aryloxy, arylalkoxy, —OCF₃, —OCO-alkyl, —OCO-alkylamino, —OCO-alkylamido, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b},
wherein Rₐ and R_{b} are independently hydrogen, alkyl, cycloalkyl, haloalkyl, aryl, aralkyl, heteroaralkyl or heteroaryl.

2. A compound of claim 1 which is of Formula II: 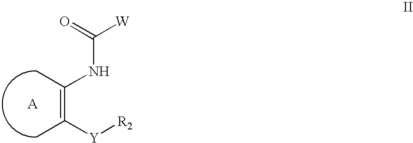
or a solvate, hydrate, tautomer or pharmaceutically acceptable salt thereof, wherein
A is
phenyl, which may be optionally substituted with one or more of -C₁₋₆ alkyl, amino, aminoalkyl, hydroxyalkyl, alkoxyalkyl, sulfonamidoalkyl, guanidinoalkyl, heteroaryl, halogen, hydroxy, —CF₃, alkoxy, aryl, aralkyl, heteroaralkyl, aryloxy, arylalkoxy, —OCF₃, —OCO-alkyl, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b};
Y is
a direct bond, —O—, or —S—;
R₂ is
alkyl, cycloalkyl, heterocyclyl, aryl or heteroaryl, each of which may be optionally substituted with one or more halogens; and
W is
furyl, imidazolyl, or pyrrolyl each of which may be optionally substituted with -C₁₋₆ alkyl, amino, aminoalkyl, hydroxyalkyl, alkoxyalkyl, heteroaryl, halogen, hydroxy, —CF₃, alkoxy, aryl, aralkyl, heteroaralkyl, aryloxy, arylalkoxy, —OCF₃, —OCO-alkyl, —OCO-alkylamino, —OCO-alkylamido, —CORₐ, —CN, —C(NH)NH₂, —COORₐ, —CONRₐR_{b}, —N(Rₐ)COR_{b}, —NO₂, —SO₂Rₐ, —SO₃Rₐ or —SO₂NRₐR_{b},
wherein Rₐ and R_{b} are independently hydrogen, alkyl, cycloalkyl, haloalkyl, aryl, aralkyl, heteroaralkyl or heteroaryl.

3. A compound of claim 2 wherein:
A is
phenyl;
Y is
a direct bond, —O—, or —S—;
R₂ is
alkyl, cycloalkyl, heterocyclyl, aryl or heteroaryl, each of which may be optionally substituted with one or more halogens; and
W is
furyl which may be optionally substituted with —CN, or —NO₂.

4. A compound according to claim 3, wherein
A is
phenyl;
Y is
a direct bond;
R₂ is
cycloalkyl, heterocyclyl, aryl or heteroaryl;
W is
cyanofuryl.

5. A compound of claim 4, which is one of
5-Cyano-furan-2-carboxylic acid (2-cyclohex-1-enyl-phenyl)-amide,
5-Cyano-furan-2-carboxylic acid biphenyl-2-ylamide,
5-Cyano-furan-2-carboxylic acid [2-(5-methyl-furan-2-yl)-phenyl]-amide,
5-Cyano-furan-2-carboxylic acid (2-furan-2-yl-phenyl)-amide,
5-Cyano-furan-2-carboxylic acid (2-thiophen-2-yl-phenyl)-amide,
5-Cyano-furan-2-carboxylic acid (2-thiophen-3-yl-phenyl)-amide,
5-Cyano-furan-2-carboxylic acid (2-furan-3-yl-phenyl)-amide,
5-Cyano-furan-2-carboxylic acid (2-cyclohexyl-phenyl)-amide,
5-Cyano-furan-2-carboxylic acid [2-(1H-pyrazol-3-yl)-phenyl]-amide,
and pharmaceutically acceptable salts thereof.

6. A compound according to claim 3, wherin
A is
phenyl;
Y is
—O— or S—;
R₂ is
alkyl, which is optionally substituted with up to five halogens;
W is
nitrofuryl.

7. A compound of claim 6, which is one of
5-nitro-furan-2-carboxylic acid [2-(2-chloro-1,1, 2-trifluoro-ethylsulfanyl)-phenyl]-amide,
5-nitro-furan-2-carboxylic acid (2-ethoxyphenyl)-amide,
and pharmaceutically acceptable salts thereof.

8. A pharmaceutical composition, comprising a compound of claim 1 and a pharmaceutically acceptable carrier.

9. A method for inhibiting protein tyrosine kinase activity, comprising contacting the kinase with an effective inhibitory amount of at least one compound of claim 1.

10. A method according to claim 8, wherein the protein tyrosine kinase is c-fms.

11. A method of treating inflammation in a mammal, comprising administering to the mammal a therapeutically effective amount of at least one compound of claim 1.

12. A method of treating cancer in a mammal, comprising administering to the mammal a therapeutically effective amount of at least one compound of claim 1.

13. A method of treating cardiovascular disease in a mammal, comprising administering to the mammal a therapeutically effective amount of at least one compound of claim 1.

14. A method of treating glomerulonephritis, rheumatoid arthritis, inflammatory bowel disease, prosthesis failure, sarcoidosis, congestive obstructive pulmonary disease, pancreatitis, HIV infection, psoriasis, diabetes, tumor related angiogenesis, restenosis, schizophrenia or Alzheimer's dementia in a mammal, comprising administering to the mammal a therapeutically effective amount of at least one compound of claim 1.

15. A pharmaceutical dosage form comprising a pharmaceutically acceptable carrier and from about 0.5 mg to about 10 g of at least one compound of claim 1.

16. A dosage form according to claim 15 adapted for parenteral or oral administration.
